(19) United States
(12) Patent Application Publication
(10) Pub. No.: US 2026/0128166 A1
(43) Pub. Date: May 7, 2026

(54) ULTRASOUND DIAGNOSTIC APPARATUS AND ULTRASOUND DIAGNOSTIC PROGRAM

(71) Applicant: FUJIFILM Corporation, Tokyo (JP)

(72) Inventor: Takuya OGAWA, Tokyo (JP)

(73) Assignee: FUJIFILM Corporation, Tokyo (JP)

(21) Appl. No.: 19/378,342

(22) Filed: Nov. 4, 2025

(30) Foreign Application Priority Data
Nov. 5, 2024 (JP) 2024-193400

(51) Int. Cl. G16H 40/40

(52) U.S. Cl. CPC G16H 40/40

(57) ABSTRACT

An ultrasound diagnostic apparatus includes an information processing unit. The information processing unit executes each of a plurality of inspection processes determined in advance for the ultrasound diagnostic apparatus based on a schedule in which an execution order is determined in advance for each of the inspection processes. The information processing unit generates report data indicating a result obtained for each of the inspection processes.

### CROSS-REFERENCE TO RELATED APPLICATION

This application claims the priority benefit of Japan application serial no. 2024-193400, filed on November 5, 2024. The entirety of the above-mentioned patent application is hereby incorporated by reference herein and made a part of this specification.

### BACKGROUND OF THE INVENTION

### 1. Field of the Invention

The present disclosure relates to an ultrasound diagnostic apparatus and an ultrasound diagnostic program, and particularly relates to inspection of an ultrasound diagnostic apparatus.

### 2. Description of the Related Art

An ultrasound diagnostic apparatus that transmits ultrasonic waves to a subject and receives the ultrasonic waves reflected by the subject to acquire image data of tissue inside the subject is used. In general, a plurality of predetermined functions of the ultrasound diagnostic apparatus are regularly inspected.

JP2011-36412A describes an ultrasound diagnostic apparatus that acquires deterioration degree information of an ultrasound probe together with identification information of the ultrasound probe. Examples of the deterioration degree information include a cumulative energization time, a cumulative usage time, and a cumulative applied voltage. JP2001-231780A describes that an automatic diagnosis of a state of an ultrasound diagnostic apparatus, which is performed by a sequence program set in advance. The sequence program is stored in a controller of the ultrasound diagnostic apparatus.

In addition, "Equipment Maintenance," Japanese Society of Sonographers, Internet <https://www.jss.org/committee/standard/04.html> describes maintenance management of an ultrasound diagnostic apparatus.

### SUMMARY OF THE INVENTION

In general, for the ultrasound diagnostic apparatus, each function is individually inspected by an operation of a user. However, a frequency and a condition of the inspection vary depending on the function of the ultrasound diagnostic apparatus. Consequently, significant effort may be expended on the work of individually managing the procedures for inspecting each function or the inspection results.

An object of the present disclosure is to simplify inspection of an ultrasound diagnostic apparatus.

The present disclosure provides an ultrasound diagnostic apparatus comprising: a processor configured to: execute each of a plurality of inspection processes determined in advance for the ultrasound diagnostic apparatus based on a schedule in which an execution order is determined in advance for each of the inspection processes; and generate report data indicating a result obtained for each of the inspection processes.

In one embodiment, the schedule defines an execution time slot for each inspection process such that a first inspection process included in one of a plurality of processing systems executed in parallel and a second inspection process included in another of the plurality of processing systems are executed, and a plurality of the first inspection processes are executed while one second inspection process is being executed.

In one embodiment, the schedule defines an execution time slot for each inspection process such that a first inspection process included in one of a plurality of processing systems executed in parallel and a second inspection process included in another of the plurality of processing systems are executed, while a preceding second inspection process is being executed, a preceding first inspection process is executed, and further, a part of a subsequent first inspection process is executed, and in association with execution of the preceding second inspection process and execution of a subsequent second inspection process, a remaining part of the subsequent first inspection process is executed while the subsequent second inspection process is being executed.

In one embodiment, the schedule defines an execution condition based on an execution frequency for each inspection process included in each of a plurality of processing systems executed in parallel, and the processor is configured to execute the inspection process in a case in which the execution condition is satisfied.

In one embodiment, the processor is configured to operate based on the schedule in a case in which power is supplied to the ultrasound diagnostic apparatus and a startup process is executed.

In one embodiment, the processor is configured to, based on the report data, cause a printer connected to the ultrasound diagnostic apparatus to create a report or cause a display unit connected to the ultrasound diagnostic apparatus to display the report.

The present disclosure further provides a storage medium storing an ultrasound diagnostic program loaded into an ultrasound diagnostic apparatus including a processor, the ultrasound diagnostic program causing the processor to execute a process comprising: executing each of a plurality of inspection processes determined in advance for the ultrasound diagnostic apparatus based on a schedule in which an execution order is determined in advance for each of the inspection processes; and generating report data indicating information obtained by each of the plurality of inspection processes.

According to the present disclosure, it is possible to simplify the inspection of the ultrasound diagnostic apparatus.

### BRIEF DESCRIPTION OF THE DRAWINGS

FIG. 1 is a diagram illustrating a configuration of an ultrasound diagnostic apparatus according to an embodiment of the present disclosure.

FIG. 2 is a diagram illustrating a table in which an example of an inspection frequency and an example of a processing time are associated with each inspection item.

FIG. 3 is a diagram illustrating a schedule of an inspection process.

FIG. 4 is a diagram illustrating a second example of the schedule of the inspection process.

FIG. 5 is a diagram illustrating a configuration of an ultrasound diagnostic system according to an application embodiment.

### DESCRIPTION OF THE PREFERRED EMBODIMENTS

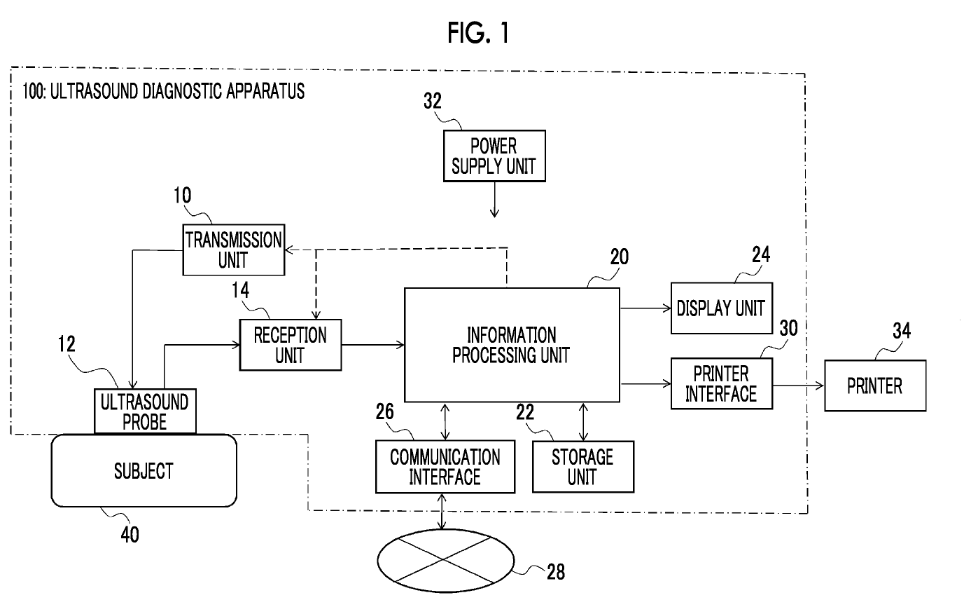

An embodiment of the present disclosure will be described with reference to the accompanying drawings. The same components illustrated in a plurality of drawings are denoted by the same reference numerals, and their description is simplified. FIG. 1 illustrates a configuration of an ultrasound diagnostic apparatus 100 according to the embodiment of the present disclosure. The ultrasound diagnostic apparatus 100 comprises a transmission unit 10, an ultrasound probe 12, a reception unit 14, an information processing unit 20, a display unit 24, a communication interface 26, a storage unit 22, and a power supply unit 32.

Each of the transmission unit 10, the ultrasound probe 12, the reception unit 14, the information processing unit 20, the display unit 24, the communication interface 26, the storage unit 22, and the power supply unit 32 may be hardware composed of an electrical circuit element or the like. The information processing unit 20 may be configured by a computer that executes a program stored in advance. The power supply unit 32 may comprise a battery, or may receive power supplied from a power supply network such as a commercial power supply. The power supply unit 32 supplies power to the ultrasound diagnostic apparatus 100. In a case in which the power supply unit 32 comprises the battery, the ultrasound diagnostic apparatus 100 may be a portable type.

The ultrasound probe 12 comprises a plurality of ultrasound transducers. The transmission unit 10 outputs a transmission signal to each ultrasound transducer included in the ultrasound probe 12 under the control of the information processing unit 20. The ultrasound probe 12 transmits ultrasonic waves based on the transmission signal output from the transmission unit 10. Each ultrasound transducer included in the ultrasound probe 12 receives reflected ultrasonic waves reflected in the subject 40, converts the reflected ultrasonic waves into a reception signal, which is an electrical signal, and outputs the reception signal to the reception unit 14.

The reception unit 14 executes, under control of the information processing unit 20, combination processing such as phase-aligned summation on the reception signals output from the respective ultrasound transducers to generate an analysis signal, and outputs the analysis signal to the information processing unit 20. The information processing unit 20 generates ultrasound image data, such as B-mode image data, Doppler waveform data, and color Doppler image data, based on the analysis signal, and displays the ultrasound image based on the ultrasound image data on the display unit 24. The display unit 24 may be a display such as a liquid-crystal display, an organic EL display, or a CRT display.

Here, the B-mode image data may be data representing a B-mode image of an observation cross section of the subject 40. The Doppler waveform data may be data representing a graph with time on a horizontal axis and blood flow velocity on a vertical axis. The color Doppler image data may be data representing a color Doppler image in which the B-mode image is colored in accordance with the blood flow velocity.

The storage unit 22 may be a storage, such as a hard disk mounted on the ultrasound diagnostic apparatus 100. In addition, a storage such as a hard disk, a memory card, or a USB memory, which is provided outside the ultrasound diagnostic apparatus 100 and is connected to the information processing unit 20, may be used instead of or together with the storage unit 22.

The communication interface 26 connects the information processing unit 20 to a communication network 28. The communication network 28 may be a local area network. The local area network may be connected to a communication network such as the Internet, which is available to the public, via a computer constituting the local area network. Further, the communication network 28 may be a communication network available to the public. That is, the communication interface 26 may connect the information processing unit 20 to a communication network available to the public.

The information processing unit 20 may perform an operation of controlling the ultrasound diagnostic apparatus 100 by executing a program stored in the storage unit 22 or a program acquired from the communication network 28 via the communication interface 26.

A printer interface 30 is connected to the information processing unit 20, and a printer 34 is connected to the printer interface 30. The information processing unit 20 outputs text data or image data to the printer 34 via the printer interface 30. The printer 34 prints a document or an image based on the text data, the image data, and the like output from the information processing unit 20 via the printer interface 30.

In the ultrasound diagnostic apparatus 100 according to the present embodiment, the information processing unit 20 executes an inspection program. The inspection program executes an inspection process on predetermined inspection items based on a predetermined schedule.

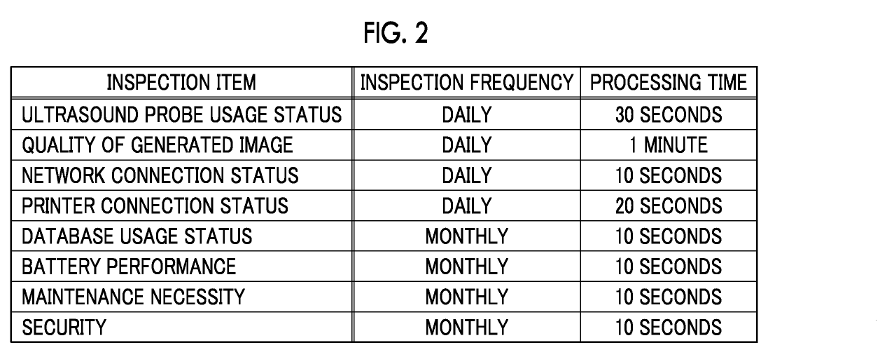

The inspection items include, for example, (i) ultrasound probe usage status, (ii) quality of generated image, (iii) network connection status, (iv) printer connection status, (v) database usage status, (vi) battery performance, (vii) maintenance necessity, and (viii) security, as illustrated in FIG. 2.

The ultrasound probe usage status is an inspection item related to the electrical stress applied to the ultrasound probe 12, such as time during which the ultrasound probe 12 is energized. The quality of the generated image is an inspection item related to the quality of the image represented by the image data generated by the information processing unit 20. The network connection status is an inspection item related to whether or not communication is established with another information processing apparatus via the communication network 28. The printer connection status is an inspection item related to whether or not the printer 34 is connected via the printer interface 30. The database usage status is an inspection item related to a usage amount of a database. Here, the database refers to database referred to during the operation of the ultrasound diagnostic apparatus 100. The database may be stored in the computer connected to the storage unit 22 or the communication network 28.

The battery performance is an inspection item in a case in which the power supply unit 32 comprises the battery. In the inspection process for the inspection item, battery evaluation information such as state of health (SOH) is acquired. The maintenance necessity is an inspection item related to whether or not there is a portion that requires maintenance. The security is an inspection item related to a security program installed in the information processing unit 20. Details of each inspection item will be described later.

FIG. 2 illustrates that an example of an inspection frequency and an example of a processing time are associated with each inspection item. The inspection frequency of each of the ultrasound probe usage status, the quality of the generated image, the network connection status, and the printer connection status is set to daily. In addition, the inspection frequency of each of the database usage status, the battery performance, the maintenance necessity, and the security is set to monthly.

The processing time is set to 1 minute for the inspection process for the quality of the generated image, 30 seconds for the inspection process for the ultrasound probe usage status, 20 seconds for the inspection process for the printer connection status, and 10 seconds for the other inspection processes. The inspection frequency and the processing time of each inspection item are not limited to those illustrated in FIG. 2 and may be selected as appropriate.

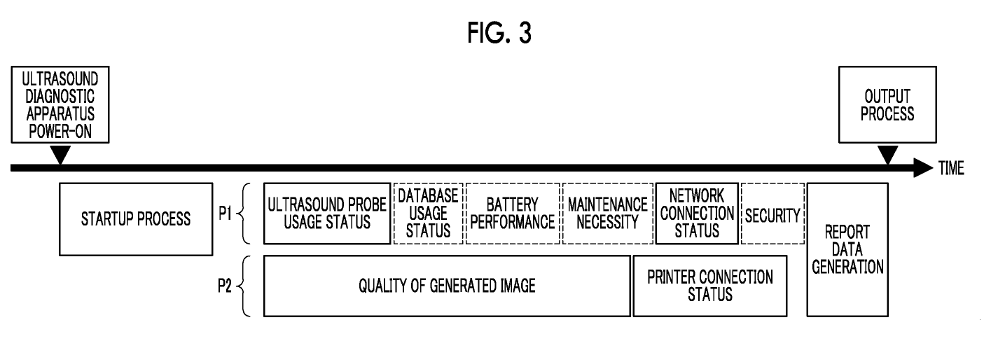

FIG. 3 illustrates an example of the schedule of the inspection process executed by the information processing unit 20 in accordance with the inspection program in the form of a timing chart. Data indicating the schedule of the inspection process may be stored in the storage unit 22 and may be read out from the storage unit 22 in a case in which the information processing unit 20 executes the inspection program. The information processing unit 20 executes a startup process in a case in which the power supply unit 32 supplies power to the ultrasound diagnostic apparatus 100. The startup process includes a process of setting each adjustment value required for acquiring the ultrasound image in order to make the ultrasound diagnostic apparatus 100 ready for use.

In the schedule illustrated in FIG. 3, a first processing system P1 and a second processing system P2 are defined as two processing systems, and the first processing system P1 and the second processing system P2 are executed in parallel by time-division processing. In the first processing system P1, an execution time slot for each inspection process is defined such that the inspection process is executed in order for the ultrasound probe usage status, the database usage status, the battery performance, the maintenance necessity, the network connection status, and the security. In the second processing system P2, an execution time slot is defined such that the inspection process is executed in order for the quality of the generated image and the printer connection status. Here, the execution time slot refers to a time slot allocated to execute a specific inspection process.

As illustrated in FIG. 2, the inspection process for the ultrasound probe usage status, the quality of the generated image, the network connection status, and the printer connection status is executed at a daily frequency. Meanwhile, the inspection process for the database usage status, the battery performance, the maintenance necessity, and the security is executed at a monthly frequency. In FIG. 3, the execution time slot of the inspection process executed at the daily frequency is indicated by a solid-line rectangle, and the execution time slot of the inspection process executed at the monthly frequency is indicated by a dashed-line rectangle.

The inspection process executed at the daily frequency and the inspection process executed at the monthly frequency are not necessarily executed together. However, for simplification of the description, a process executed by the information processing unit 20 in a case in which all the inspection items are executed will be described here.

Each execution time slot in which the inspection process for the quality of the generated image is executed is longer than the execution time slot in which the inspection process for the ultrasound probe usage status is executed, the execution time slot in which the inspection process for the database usage status is executed, the execution time slot in which the inspection process for the battery performance is executed, and the execution time slot in which the inspection process for the maintenance necessity is executed. In addition, the execution time slot in which the inspection process for the printer connection status is executed is longer than the execution time slot in which the inspection process for the network connection status is executed and the execution time slot in which the inspection process for the security is executed.

The information processing unit 20 executes the inspection process for the quality of the generated image included in the second processing system P2 and executes the inspection process for the ultrasound probe usage status included in the first processing system P1, and then executes the inspection processes for the database usage status, the battery performance, and the maintenance necessity included in the first processing system P1.

After the inspection process for the quality of the generated image has ended, while the inspection process for the printer connection status is being executed, the information processing unit 20 ends the inspection process for the maintenance necessity, starts the inspection process for the network connection status, and ends the inspection process for the network connection status. The information processing unit 20 starts the inspection process for the security after the inspection process for the network connection status has ended. The information processing unit 20 ends the inspection process for the printer connection status while the inspection process for the security is being executed, and then ends the inspection process for the security.

As described above, the execution time slot for each inspection process is defined such that, while the inspection process for the quality of the generated image is being executed as one inspection process (second inspection process) included in the second processing system P2, the inspection processes for the ultrasound probe usage status, the database usage status, and the battery performance are executed as a plurality of inspection processes (first inspection processes) included in the first processing system P1.

In addition, the execution time slot for each inspection process is defined such that, while the inspection process for the quality of the generated image is being executed as a preceding inspection process (preceding second inspection process) included in the second processing system P2, the inspection process for the battery performance is executed as a preceding inspection process (preceding first inspection process) included in the first processing system P1, and further, a part of the inspection process for the maintenance necessity is executed as a part of a subsequent inspection process (subsequent first inspection process) included in the first processing system P1. In addition, the execution time slot for each inspection process is defined such that, in association with execution of the inspection process for the quality of the generated image as the preceding inspection process (preceding second inspection process) included in the second processing system P2 and execution of the inspection process for the printer connection status as the subsequent inspection process (subsequent second inspection process), a remaining part of the inspection process for the maintenance necessity as a remaining part of the subsequent inspection process included in the first processing system P1 is executed while the inspection process for the printer connection status is being executed. That is, the execution time slot for each inspection process is defined such that the inspection process for the maintenance necessity is executed in a time slot that spans both the time slot in which the inspection process for the quality of the generated image is executed and the time slot in which the inspection process for the printer connection status is executed. By executing the process via the information processing unit 20 based on the schedule, the inspection process is simplified, and thus the time required for the plurality of inspection processes is shortened.

Here, the operation of executing the inspection process for all the inspection items with a monthly inspection frequency has been described. The schedule may set different days for executing the inspection process for the plurality of inspection items with a monthly inspection frequency.

In the schedule, the execution order of each inspection process is defined for each processing system, but the information processing unit 20 may execute the inspection process for the inspection item for which the execution time interval determined by the inspection frequency has elapsed from the last execution of the inspection process.

That is, the schedule may define an execution condition based on the execution frequency for each inspection process included in each of the plurality of processing systems, and the information processing unit 20 may execute the inspection process in the execution time slot defined for each inspection process in a case in which the execution condition is satisfied.

More specifically, in the schedule, for each inspection item, a date and time when the inspection process is last executed may be recorded as a final execution time. The information processing unit 20 may refer to the record of the final execution time and may execute the inspection process for the inspection item for which 24 hours has elapsed from the last execution of the inspection process, for the inspection item with a daily inspection frequency. Similarly, the information processing unit 20 may execute the inspection process for the inspection item for which one month has elapsed from the last execution of the inspection process, for the inspection item with a monthly inspection frequency.

Here, the inspection item with a daily inspection frequency and the inspection item with a monthly inspection frequency have been described. The inspection frequency of each inspection item may be at a predetermined time interval, for example, every week or every year. In this case, the information processing unit 20 refers to the record of the final execution time and executes the inspection process for the inspection item for which the execution time interval determined by the inspection frequency has elapsed from the last execution of the inspection process.

After the inspection process for each inspection item defined in the schedule is executed, the information processing unit 20 generates report data. In accordance with the schedule illustrated in FIG. 3, the information processing unit 20 generates the report data after ending the inspection process for the security.

The report data is data indicating the result of each inspection process. The report data may be text data, image data, data obtained by combining text data and image data, data in CSV format, data displayed by a spreadsheet program, or the like.

After the report data is generated, the information processing unit 20 executes an output process on the report data. The output process may be a process of converting the report data into printing report data for printing the result of each inspection process on paper and outputting the printing report data to the printer 34 via the printer interface 30. The printer 34 may create a report by printing the result of each inspection process on the paper based on the printing report data.

The output process may be a process of converting the report data into a video signal for displaying the result of each inspection process on the display unit 24 and outputting the video signal to the display unit 24. The display unit 24 may display the report as the result of each inspection process based on the video signal.

The output process may include a process of storing the report data in the storage unit 22.

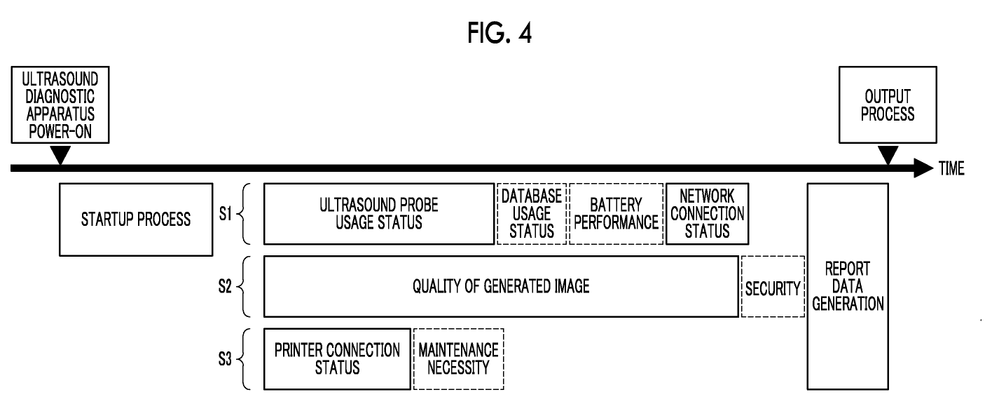

FIG. 4 illustrates a second example of the schedule of the inspection process in the form of a timing chart. In the schedule illustrated in FIG. 4, a first processing system S1, a second processing system S2, and a third processing system S3 are defined as three processing systems, and the first processing system S1 to the third processing system S3 are executed in parallel by time-division processing.

In the first processing system S1, the execution time slot is defined such that the inspection process is executed in order for the ultrasound probe usage status, the database usage status, the battery performance, and the network connection status. In the second processing system S2, the execution time slot is defined such that the inspection process is executed in order for the quality of the generated image and the security. In the third processing system S3, the execution time slot is defined such that the inspection process is executed in order for the printer connection status and the maintenance necessity.

In the schedule illustrated in FIG. 4, the execution order and the execution time slot of each inspection process are defined for each processing system, but the information processing unit 20 may execute the inspection process for the inspection item for which the execution time interval determined by the inspection frequency has elapsed from the last execution of the inspection process. Here, for simplification of the description, a case will be described in which all the inspection items are executed.

The time during which the inspection process for the quality of the generated image is executed is longer than the times during which the other inspection processes are executed. The information processing unit 20 executes the inspection process for the quality of the generated image included in the second processing system S2 and executes the inspection process for the ultrasound probe usage status included in the first processing system S1, and then executes the inspection processes for the database usage status, the battery performance, and the network connection status.

After the inspection process for the quality of the generated image has ended, the information processing unit 20 ends the inspection process for the network connection status when the inspection process for the security is executed. That is, the information processing unit 20 ends the inspection process for the network connection status while the inspection process for the security is being executed.

As described above, the execution time slot is defined such that, while the inspection process for the quality of the generated image is being executed as one inspection process included in the second processing system S2, the inspection processes for the ultrasound probe usage status, the database usage status, and the battery performance are executed as the plurality of inspection processes included in the first processing system S1.

In addition, the execution time slot is defined such that, while the inspection process for the quality of the generated image is being executed as the preceding inspection process included in the second processing system S2, the inspection process for the battery performance is executed as the preceding inspection process included in the first processing system S1, and further, a part of the inspection process for the network connection status is executed as a part of the subsequent inspection process included in the first processing system S1. In addition, an execution time slot is defined such that, in association with the execution of the inspection process for the quality of the generated image as the preceding inspection process included in the second processing system S2 and the execution of the inspection process for the security as the subsequent inspection process, a remaining part of the inspection process for the network connection status is executed as a remaining part of the subsequent inspection process included in the first processing system S1 while the inspection process for the security is executed. That is, the inspection process for the network connection status is executed in a time slot that spans both the time slot in which the inspection process for the quality of the generated image is executed and the time slot in which the inspection process for the security is executed. By executing the process via the information processing unit 20 based on the schedule, the inspection process is simplified, and thus the time required for the plurality of inspection processes is shortened.

While the inspection process for the quality of the generated image is being executed, the information processing unit 20 executes the inspection processes for the printer connection status and the maintenance necessity included in the third processing system S3. After the inspection process for each inspection item defined in the schedule is executed, the information processing unit 20 generates the report data. In accordance with the schedule illustrated in FIG. 4, the information processing unit 20 generates the report data after ending the inspection process for the security.

In the above description, the embodiment has been described in which the inspection process for each inspection item based on the schedule is executed after the power is supplied to the ultrasound diagnostic apparatus 100 and the startup process is executed. The inspection process for each inspection item based on the schedule may be executed on a date and time set by the user. For example, the inspection process for each inspection item based on the schedule may be executed, in a case in which the power of the ultrasound diagnostic apparatus 100 is on, on a date and time set by the user. In addition, the inspection process for each inspection item based on the schedule may be executed in response to the operation of the user.

Hereinafter, each inspection item will be specifically described. The ultrasound probe usage status is represented, for example, by time-based metrics indicative of electrical stress such as a cumulative energization time of the ultrasound probe 12, a cumulative usage time, and a cumulative applied voltage within a recent predetermined time (for example, 24 hours). The cumulative energization time of the ultrasound probe 12 is defined, for example, as the cumulative total value of times during which power is supplied to the ultrasound probe 12, starting from when the ultrasound probe 12 is connected to the ultrasound diagnostic apparatus 100. The cumulative usage time of the ultrasound probe 12 is defined, for example, as the cumulative total value of times during which the transmission unit 10 outputs the transmission signal to any of the plurality of ultrasound transducers included in the ultrasound probe 12, starting from when the ultrasound probe 12 is connected to the ultrasound diagnostic apparatus 100. The cumulative applied voltage within the recent predetermined time is defined as a time integral value of the power supply voltage applied to the ultrasound probe 12. The cumulative energization time of the ultrasound probe 12, the cumulative usage time, the cumulative applied voltage within the recent predetermined time, and the like may be measured by the information processing unit 20 while the power is supplied from the power supply unit 32 to the ultrasound diagnostic apparatus 100, and the measurement results may be stored in the storage unit 22.

The information processing unit 20 executes the inspection process for the ultrasound probe usage status, generates ultrasound probe information including at least one of the cumulative energization time of the ultrasound probe 12, the cumulative usage time, or the cumulative applied voltage within the recent predetermined time as the time-based metrics indicative of the electrical stress, and stores the ultrasound probe information in the storage unit 22. The ultrasound probe information indicates a degree of the electrical stress applied to the ultrasound probe 12.

The quality of the generated image may be represented by, for example, an evaluation value indicating a difference from reference image data. That is, in the inspection process for the quality of the generated image, the information processing unit 20 acquires test ultrasound image data in a case in which the ultrasound probe 12 is in a test state. The test state may be a state in which the ultrasound probe 12 is in contact with a phantom or a water bag simulating the subject 40. Further, the test state may be a state in which the ultrasound probe 12 is released in a free space or a state in which the ultrasound probe 12 is held by a predetermined holder.

The information processing unit 20 executes the inspection process for the quality of the generated image, acquires an image evaluation value representing a difference between the test ultrasound image data and reference image data acquired in advance, and stores the image evaluation value in the storage unit 22. The image evaluation value may be a value representing a degree of similarity between an image represented by the generated image generated by the information processing unit 20 and an image represented by the reference image data. The image evaluation value may be, for example, a correlation value between the image represented by the test ultrasound image data and the image represented by the reference image data. The correlation value indicates the degree of similarity between two images. The greater the degree of similarity, the higher the quality of the generated image.

The network connection status may be represented by, for example, whether or not communication is established with a predetermined connection destination in the communication network 28, such as the Internet. In the inspection process for the network connection status, the information processing unit 20 performs communication with a predetermined connection destination in the communication network 28 via the communication interface 26. The information processing unit 20 acquires, from the predetermined connection destination, communication establishment information indicating that normal communication is being performed, such as acknowledgment packets, and stores the information in the storage unit 22.

The printer connection status may be represented by, for example, whether or not the printer 34 is connected to the information processing unit 20 via the printer interface 30. In the inspection process for the printer connection status, the information processing unit 20 transmits test data to the printer interface 30 and determines whether or not response data to the test data is output from the printer interface 30 to the information processing unit 20. In a case in which the response data is output from the printer interface 30, the information processing unit 20 stores printer connection information indicating that the printer 34 is connected in the storage unit 22. In a case in which the response data is not output from the printer interface 30, the information processing unit 20 stores the printer connection information indicating that the printer 34 is not connected in the storage unit 22.

The database usage status may be represented by, for example, a usage amount of the database. The database may be stored in the computer connected to the storage unit 22 or the communication network 28. The database may include reference data regarding human tissues, and data required for a preset function that sets the state of the ultrasound diagnostic apparatus 100 to a state suitable for diagnosing a specific tissue. The information processing unit 20 may measure the usage amount of the database while the power supply unit 32 supplies power to the ultrasound diagnostic apparatus 100, and store the measurement result in the storage unit 22. The usage amount of the database may be measured by an information amount (in bytes) of data used after the power is supplied, a search time of searching for specific data in the database, an information amount of data used within a recent certain time, or the like. In the inspection process for the database usage status, the information processing unit 20 generates database determination information indicating whether or not the usage amount of the database is equal to or greater than a specified value, and stores the database determination information in the storage unit 22. The information processing unit 20 may store the database determination information including the usage amount of the database in the storage unit 22.

The inspection process for the battery performance is executed in a case in which the power supply unit 32 comprises the battery. The battery evaluation information indicating the battery performance may include at least one of the cumulative energization time, the cumulative usage time, or the state of health (SOH). The cumulative energization time may be defined as, for example, a cumulative total value of times during which the current flows through the battery, such as the time during which the battery supplies power to the ultrasound diagnostic apparatus 100, and the time during which the battery is charged. The cumulative usage time may be defined as, for example, a cumulative total value of times during which the power is supplied to the ultrasound diagnostic apparatus 100.

The information processing unit 20 may measure a cumulative total value (aggregate total value) of times during which the current flows through the battery, as the cumulative energization time. The information processing unit 20 may measure a cumulative total value (aggregate total value) of times during which the power is supplied to the ultrasound diagnostic apparatus 100 from the battery, as the cumulative usage time. The information processing unit 20 may measure a state of charge (SOC) of the battery, and further obtain a state of health (SOH). The SOC is an amount of charge of the battery expressed as a percentage, with the amount of charge at full charge taken as 100%. The SOH is a ratio, expressed as a percentage, of the present full-charge capacity to the full-charge capacity in a case in which the battery is new. Here, the full-charge capacity is defined as a maximum charge amount that can be charged.

In the inspection process for the battery performance, the information processing unit 20 generates battery performance information and stores the battery performance information in the storage unit 22. The battery performance information may include, in addition to at least one of the cumulative energization time, the cumulative usage time, or the state of health (SOH), at least any one of information on whether or not the cumulative energization time exceeds a limit value, information on whether or not the cumulative usage time exceeds a limit value, or information on whether or not the SOH is less than a reference value.

The information processing unit 20 generates security information by executing the inspection process for the security, and stores the security information in the storage unit 22. The security information may include information indicating whether or not a version of a security program installed in the information processing unit 20 is up to date. The security program may be, for example, an antivirus program. Further, the communication establishment information for a predetermined connection destination may be included. The communication establishment information may be information stored in the storage unit 22 in advance by the information processing unit 20. In the inspection process for the security, the information processing unit 20 acquires the security information and stores the security information in the storage unit 22.

The inspection process for the maintenance necessity is a process of generating maintenance information indicating a portion that requires maintenance. The information processing unit 20 generates the maintenance information based on the results of the inspection processes for the ultrasound probe usage status, the quality of the generated image, the network connection status, the printer connection status, the database usage status, and the battery performance, and stores the generated maintenance information in the storage unit 22.

In a case in which a numerical value included in the ultrasound probe information obtained by the ultrasound probe usage status inspection process is outside a specified range, the information processing unit 20 may include, in the maintenance information, information indicating that the ultrasound probe 12 requires maintenance.

In a case in which the image evaluation value obtained by the inspection process for the quality of the generated image is outside a specified range, the information processing unit 20 may include, in the maintenance information, information indicating that a component (adjustment of a setting value of a program, adjustment of an operation setting value, and the like) that generates the image data requires maintenance.

In a case in which, through the inspection process for the network connection status, it is determined that communication is not normally performed with the predetermined connection destination, the information processing unit 20 may include, in the maintenance information, information indicating that the communication function requires maintenance.

In a case in which, through the inspection process for the printer connection status, it is determined that the printer 34 is not connected, the information processing unit 20 may include, in the maintenance information, information indicating that the printer 34 requires maintenance.

In a case in which the usage amount of the database obtained by the database usage status inspection process is outside a specified range, the information processing unit 20 may include, in the maintenance information, information indicating that the method of using the ultrasound diagnostic apparatus 100 needs to be checked.

In a case in which a numerical value included in the battery evaluation information obtained by the battery performance inspection process is outside a specified range, the information processing unit 20 may include, in the maintenance information, information indicating that the battery requires maintenance, such as replacement of the battery.

The report data generated by the information processing unit 20 after all the inspection processes have ended may be data including the following information.

(i) Ultrasound probe information obtained by inspection process for ultrasound probe usage status

(ii) Image evaluation value obtained by inspection process for quality of generated image

(iii) Communication establishment information obtained by inspection process for network connection status

(iv) Printer connection information obtained by inspection process for printer connection status

(v) Database determination information obtained by inspection process for database usage status

(vi) Battery evaluation information obtained by inspection process for battery performance

(vii) Maintenance information obtained by inspection process for maintenance necessity

(viii) Security information obtained by inspection process for security

The report data may be, for example, all or a part of "Ultrasound Diagnostic Apparatus System Management Inspection Checklist" established by the Japanese Society of Sonographers. The "Ultrasound Diagnostic Apparatus System Management Inspection Checklist" is illustrated in "Equipment Maintenance," Japanese Society of Sonographers, Internet <https://www.jss.org/committee/standard/04.html>.

In the present embodiment, each processing is executed by any computer. Moreover, any computer may execute these kinds of processing by a processor as hardware, a program as software, or a combination thereof. In such a case, the processor is configured to execute various kinds of processing in the present embodiment in cooperation with the program, and may function as each unit or each means in the present embodiment. Further, the execution order of the processing by the processor is not limited to the above-described order, and may be changed as appropriate. Any computer may be a general-purpose computer, a computer for specific use, a workstation, or another system that can execute each processing.

The processor may be configured by one or more kinds of hardware, and the type of hardware is not limited. For example, the processor may be configured by a programmable logic device such as a central processing unit (CPU), a microprocessing unit (MPU), or a field programmable gate array (FPGA), a dedicated circuit for executing specific processing, such as an application-specific integrated circuit (ASIC), or hardware such as a graphics processing unit (GPU) or a neural processing unit (NPU). Moreover, the type of hardware may be a combination of different kinds of hardware. In a case in which the plurality of kinds of hardware are configured to execute one or more kinds of processing of a certain processor, the plurality of kinds of hardware may be present in devices physically separated from each other or may be present in the same device. In addition, in any embodiment, the order of each processing by the processor is not limited to the order described in the specification of the present application, and may be changed as appropriate. In addition, hardware is implemented in the form of an electric circuit (circuitry) in which circuit elements, such as semiconductor elements, are combined.

Furthermore, the program may be software such as firmware or microcode. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

The present disclosure is also applicable to a program and a program product. An ultrasound diagnostic program according to the above-described embodiment of the present disclosure causes the processor included in the ultrasound diagnostic apparatus 100 to execute a process comprising: executing each of a plurality of inspection processes determined in advance for the ultrasound diagnostic apparatus 100 based on a schedule in which an execution order is determined in advance for each of the inspection processes; and generating report data indicating information obtained by each of the plurality of inspection processes. The program may be stored in and provided from a storage medium such as a memory card, a USB memory, or a CD-ROM.

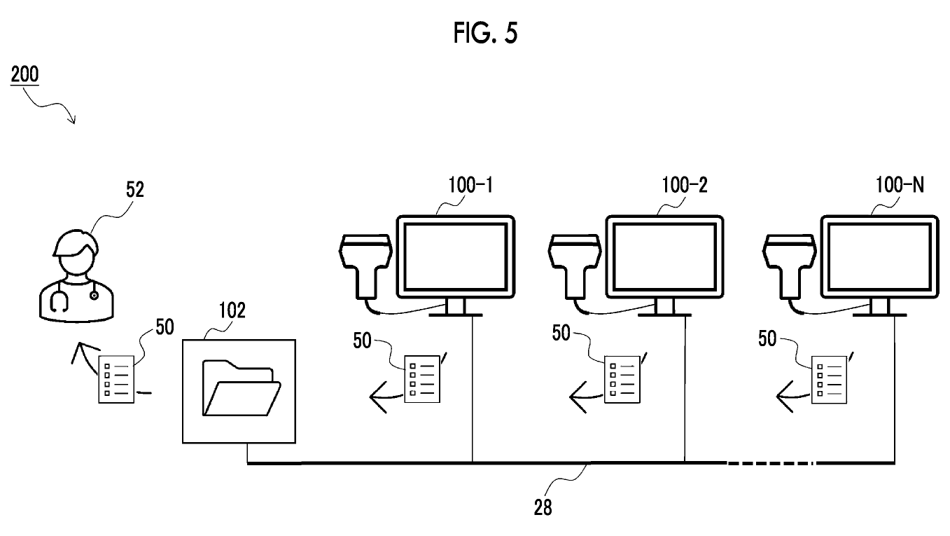

FIG. 5 illustrates a configuration of an ultrasound diagnostic system 200 according to an application embodiment of the present disclosure. The ultrasound diagnostic system 200 comprises N ultrasound diagnostic apparatuses 100-1 to 100-N, the communication network 28, and a host computer 102. The ultrasound diagnostic apparatuses 100-1 to 100-N have the same configuration as the ultrasound diagnostic apparatus 100 illustrated in FIG. 1. The ultrasound diagnostic apparatuses 100-1 to 100-N are connected to the host computer 102 via the communication network 28.

Each of the ultrasound diagnostic apparatuses 100-1 to 100-N generates report data 50, and transmits the report data 50 to the host computer 102 via the communication network 28. A user 52 may display a report based on the report data 50 generated by each of the ultrasound diagnostic apparatuses 100-1 to 100-N on the host computer 102. As a result, the user 52 can easily ascertain the states of the ultrasound diagnostic apparatuses 100-1 to 100-N.

What is claimed is:

1. An ultrasound diagnostic apparatus comprising:
a processor configured to:
execute each of a plurality of inspection processes determined in advance for the ultrasound diagnostic apparatus based on a schedule in which an execution order is determined in advance for each of the inspection processes; and
generate report data indicating a result obtained for each of the inspection processes.

2. The ultrasound diagnostic apparatus according to claim 1,
wherein the schedule defines an execution time slot for each inspection process such that
a first inspection process included in one of a plurality of processing systems executed in parallel and a second inspection process included in another of the plurality of processing systems are executed, and
a plurality of the first inspection processes are executed while one second inspection process is being executed.

3. The ultrasound diagnostic apparatus according to claim 1,
wherein the schedule defines an execution time slot for each inspection process such that
a first inspection process included in one of a plurality of processing systems executed in parallel and a second inspection process included in another of the plurality of processing systems are executed,
while a preceding second inspection process is being executed, a preceding first inspection process is executed, and further, a part of a subsequent first inspection process is executed, and
in association with execution of the preceding second inspection process and execution of a subsequent second inspection process, a remaining part of the subsequent first inspection process is executed while the subsequent second inspection process is being executed.

4. The ultrasound diagnostic apparatus according to claim 1,
wherein the schedule defines an execution condition based on an execution frequency for each inspection process included in each of a plurality of processing systems executed in parallel, and
the processor is configured to execute the inspection process in a case in which the execution condition is satisfied.

5. The ultrasound diagnostic apparatus according to claim 1,
wherein the processor is configured to operate based on the schedule in a case in which power is supplied to the ultrasound diagnostic apparatus and a startup process is executed.

6. The ultrasound diagnostic apparatus according to claim 1,
wherein the processor is configured to, based on the report data, cause a printer connected to the ultrasound diagnostic apparatus to create a report or cause a display unit connected to the ultrasound diagnostic apparatus to display the report.

7. A non-transitory computer-readable storage medium storing an ultrasound diagnostic program loaded into an ultrasound diagnostic apparatus including a processor, the ultrasound diagnostic program causing the processor to execute a process comprising:
executing each of a plurality of inspection processes determined in advance for the ultrasound diagnostic apparatus based on a schedule in which an execution order is determined in advance for each of the inspection processes; and
generating report data indicating information obtained by each of the plurality of inspection processes.
